(19) United States
(12) Patent Application Publication
(10) Pub. No.: US 2026/0083783 A1
(43) Pub. Date: Mar. 26, 2026

(54) Engineered Probiotics Expressing Anti-Inflammatory Molecules

(71) Applicant: University of Cincinnati, Cincinnati, OH (US)

(72) Inventors: Nalinikanth Kotagiri, West Chester, OH (US); Tushar Madaan, Cincinnati, OH (US)

(73) Assignee: University of Cincinnati, Cincinnati, OH (US)

(21) Appl. No.: 19/111,351

(22) PCT Filed: Sep. 13, 2023
(86) PCT No.: PCT/US23/32641

§ 371 Date: Mar. 13, 2025

Related U.S. Application Data
(60) Provisional application No. 63/406,004, filed on Sep. 13, 2022.

(51) Int. Cl. A61K 35/741; A61P 9/00; C07K 14/195; C12N 15/70; C12R 1/19

(52) U.S. Cl. CPC A61K 35/741; A61P 9/00; C07K 14/195; C12N 15/70; C12R 2001/19

(57) ABSTRACT

A genetically modified E. coli bacterium is provided where the bacterium expresses a microbial anti-inflammatory molecule (MAM) protein. In one embodiment, the bacterium is a probiotic. In another embodiment, the bacterium is modified is E. coli Nissle 1917. In one embodiment, the MAM protein has a sequence of [SEQ ID NO: 1]. In another embodiment, the bacterium expresses a plasmid having a sequence of [SEQ ID NO: 2].

### CROSS REFERENCE TO RELATED APPLICATIONS

This application is a continuation of PCT Application No. PCT/US23/32641 filed Sep. 13, 2023, which claims the benefit of the filing date of, U.S. Patent Application Ser. No. 63/406,004, filed Sep. 13, 2022, the disclosures of which are incorporated by reference herein in their entirety.

### STATEMENT REGARDING FEDERALLY SPONSORED RESEARCH OR DEVELOPMENT

This invention was made with government support under grant R21GM137321 awarded by the National Institutes of Health. The government has certain rights in the invention.

### FIELD OF THE INVENTION

This invention relates generally to engineered probiotics.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Cardiovascular disease remains the leading cause of mortality in the United States, with an annual total cost of over $500 billion. Myocardial infarction, often referred to as heart attack in lay terms, is generally caused by the decrease or cessation of blood flow to a part of the heart resulting in myocardial injury or necrosis. Risk factors for myocardial infarction include Western diet, diabetes, sedentary lifestyle, hypertension, atherosclerosis, high body mass index, etc. all of which are prevalent in the United States, resulting in a large number of people being predisposed to the disease. Data from 2005-2014 reveals that there are an estimated 605,000 new and 200,000 recurrent cases of myocardial infarction each year in the United States with an American suffering from a heart attack every 39 seconds. In addition to the extremely high morbidity and mortality associated with the disease, MIs are one of the most expensive conditions to treat within the United States resulting in a significant financial burden on the patients.

Conventional oral drug delivery systems are plagued with numerous problems such as low patient compliance, non-targeted treatment, inability to deliver protein, peptides, and biological drugs, and linear therapeutic effect irrespective of disease state or severity. Protein and biologics-based drugs are gaining ground as therapeutics of choice for numerous difficult to treat illnesses with eight of the top ten drugs by revenue belonging to this category. Therefore, there is a significant unmet need for developing pioneering drug delivery systems that can orally deliver biologics and protein-based drugs. Such a delivery system will not only allow us to target several disease conditions by repurposing existing drugs but would also encourage the development of new therapeutics that can be delivered using such a platform.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention.

In an embodiment of the invention, a genetically modified E. coli bacterium is provided where the bacterium expresses a microbial anti-inflammatory molecule (MAM) protein. In one embodiment, the bacterium is a probiotic. In another embodiment, the bacterium is modified is E. coli Nissle 1917. In one embodiment, the MAM protein has a sequence of [SEQ ID NO: 1]. In another embodiment, the bacterium expresses a plasmid having a sequence of [SEQ ID NO: 2].

In another embodiment of the invention, a pharmaceutical composition is provided that includes a genetically modified E. coli bacterium wherein the bacterium expresses a microbial anti-inflammatory molecule (MAM) protein and a pharmaceutically acceptable excipient. In one embodiment, the bacterium in the pharmaceutical composition is a probiotic. In another embodiment, the bacterium in the pharmaceutical composition is modified is E. coli Nissle 1917. In one embodiment, the MAM protein has a sequence of [SEQ ID NO: 1]. In another embodiment, the bacterium in the pharmaceutical composition expresses a plasmid having a sequence of [SEQ ID NO: 2].

In another embodiment of the invention, a plasmid comprising the sequence of [SEQ ID NO: 2] is provided.

In another embodiment of the invention, a method of reducing cardiac inflammation in a subject who has experienced a myocardial infarction is provided. The method involves administering a pharmaceutically effective amount of a genetically modified E. coli bacterium to the subject, where the bacterium expresses a microbial anti-inflammatory molecule (MAM) protein. In one embodiment, the bacterium is a probiotic. In another embodiment, the bacterium that is modified is E. coli Nissle 1917. In one embodiment, the MAM protein has a sequence of [SEQ ID NO: 1]. In one embodiment, the bacterium expresses a plasmid having a sequence of [SEQ ID NO: 2]. In another embodiment, a pharmaceutical composition including the genetically modified E. coli bacterium and a pharmaceutically acceptable excipient is administered to the patient. In one embodiment, the genetically modified E. coli bacterium is administered orally.

### BRIEF DESCRIPTION OF THE DRAWINGS

The objects and advantages of the disclosed invention will be further appreciated in light of the following detailed descriptions and drawings in which:

FIG. 1 is a graph showing that mouse heart weight to body ratio indicates mice given EcN Lux have significantly higher cardiac hypertrophy and fibrosis compared to mice given EcN MAM.

FIG. 2A is an image of a Masson's trichrome stain of a heart of an MI mouse given E. coli Nissle 1917 expressing MAM.

FIG. 2B is an image of a Masson's trichrome stain of a heart of an MI mouse given E. coli Nissle 1917 pAKgfplux1.

FIG. 3A is a schematic of the gene circuit for MAM protein synthesis.

FIG. 3B is an immunoblot of bacterial protein extract demonstrating that the engineered EcN-MAM construct produces F. prausnitzii derived MAM protein.

FIG. 4A is a graph of flow cytometric analysis showing that EcN-MAM treated mice have significantly fewer numbers of monocytes at 48 h timepoint.

FIG. 4B is a graph of flow cytometric analysis showing that EcN-MAM treated mice have significantly fewer numbers of macrophages at 48 h timepoint.

FIG. 4C is a graph showing MI mice treated with EcN-MAM had significantly lower numbers of neutrophils compared to EcN-Lux and PBS treated controls.

FIG. 4D is a graph showing MI mice treated with EcN-MAM had significantly lower numbers of activated neutrophils compared to EcN-Lux and PBS treated controls.

FIG. 4E is an image of immunoblots of Occludin and ZO-1 showing that MI mice treated with EcN-MAM have significantly higher levels of intestinal tight junction proteins vs. mice given EcN-Lux or PBS.

FIG. 4F is a graph showing MI mice given EcN-MAM have significantly lower levels of Serum LBP, indicating less leakage of gut bacteria into the systemic circulation.

FIG. 4G is a graph showing that Serum C-Reactive Protein (CRP) levels are significantly elevated in control mice given EcN-Lux but are significantly resolved in mice given EcN-MAM or mice with antibiotic induced intestinal microbiome depletion.

### DEFINITIONS

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

The term “engineered”, as used herein, refers to a nucleic acid molecule, protein molecule, complex, substance, or entity that has been artificially designed, produced, prepared, synthesized and/or manufactured. Therefore, the engineered product is a non-naturally occurring product.

As used herein, the term “engineered bacterium” or “engineered bacterial cell” refers to a bacterial cell that has been genetically modified from its native state. For instance, an engineered bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Engineered bacterial cells of the disclosure may comprise exogenous nucleotide sequences on plasmids. Alternatively, recombinant bacterial cells may comprise exogenous nucleotide sequences stably incorporated into their chromosome.

“Probiotic”, as used herein, refers to a live, non-pathogenic microorganism, e.g., a bacterium, which can confer health benefits to a host organism. In some embodiments, the host organism is a mammal. In some embodiments, the host organism is a human. Some species, strains, and/or subtypes of non-pathogenic bacteria are currently recognized as probiotic bacteria. Examples of probiotic bacteria include, but are not limited to, Salmonella typimurium, Listeria monocytogenes, Staphylococcus epidermidis, Bifidobacterium, Bacteroides, Bacillus, Burkholderia cepacia, Propionibacterium, Fusobacterium, Campylobacter jejuni, Lactobacillus acidophilus, Klebsiella, Bacillus coagulans, Enterococcus and Streptococcus, including Streptococcus oralis. The probiotic may be a variant or a mutant strain of bacterium. Non-pathogenic bacteria may be genetically engineered to enhance or improve desired biological properties, e.g., survivability.

A “pharmaceutical composition,” as used herein, refers to a composition comprising an active ingredient (e.g., a bacterial cell, an inducer, a drug, or a detectable compound) with other components such as a physiologically suitable carrier and/or excipient.

As used herein, the term “pharmaceutically acceptable” or “pharmacologically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Moreover, for animal (e.g., human) administration, it will be understood that compositions should meet sterility, pyrogenicity, general safety and purity standards as required by the FDA Office of Biological Standards.

As used herein, the term “pharmaceutically acceptable excipient” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, disintegrating agents, binders, sweetening agents, flavoring agents, perfuming agents, protease inhibitors, plasticizers, emulsifiers, stabilizing agents, viscosity increasing agents, film forming agents, solubilizing agents, surfactants, preservative and antioxidants can also be present in the formulation. The terms such as “excipient”, “carrier”, “pharmaceutically acceptable excipient” or the like are used interchangeably herein.

As used herein, the term “plasmid” refers to a construct composed of genetic material (i.e., nucleic acid).

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As used herein, the term “pharmaceutically effective” means an amount sufficient to effect the desired change in the subject.

The present invention concerns the use of probiotic bacteria as oral drug delivery systems and their role in the treatment of myocardial infarction. Probiotic gut bacteria such as Escherichia coli Nissle 1917 (EcN) are excellent for this purpose as, when administered in appropriate quantities, they can surpass the adverse environments offered by the gastrointestinal tract and colonize the gut. Furthermore, being native to the human gut, engineered EcN can not only help ameliorate gut dysbiosis, but also produce the desired therapeutics and aid in the treatment of the targeted disease. Our studies have revealed that EcN can also colonize and proliferate in other regions of hypoxia in the body such as infarcted cardiac tissue. Therefore, the genetically engineered bacteria of the present invention shows its efficacy by colonizing the gut and producing anti-inflammatory therapeutics. The production of the therapeutic molecules within the gut ecosystem allows for the direct absorption of the drug via the intestines while the rapid multiplication of bacteria allows for sustained delivery of therapeutics. Exorbitant costs of drugs, particularly proteins, peptides, and biologicals inhibit their accessibility across the world. Additionally, a large amount of resources in terms of biomanufacturing, analysis, and extraction/purification facilities are required for drugs belonging to these classes. The use of probiotic bacteria circumvents all of these problems and costs since there is no need to obtain pure molecules from culture and therapeutics are produced in situ in the intestines.

In one embodiment, the present invention takes the novel approach of using a recombinant probiotic platform secreting a defined therapeutic protein, MAM, to target intestinal hyperpermeability and protect against excessive systemic permeation due to systemic bacterial permeation. In another embodiment, the present invention enables the oral administration of EcN-MAM, which significantly reduces post-MI cardiac inflammation and fibrosis.

### Cardiac Translocation of Gut Bacteria

Acute myocardial infarction (AMI) is a serious cardiac emergency resulting in hypoxia, and eventually necrosis in the myocardium. AMI is generally precipitated by an unstable cardiac ischemia and is a major cause of morbidity and mortality across the world. According to the National Health and Nutrition Examination Survey (NHANES) 2013-2016, the overall prevalence of myocardial infarction in US adults 20 years and above in age is 3%. Statistics indicate that approximately every 40 seconds, an American suffers from myocardial infarction with an estimated 605,000 new MI attacks and 200,000 recurrent attacks occurring annually.

Recent studies have underscored the role of the gut-heart axis, particularly the gut microbiome in the pathophysiology of myocardial infarction. Patients of MI have been found to have significantly higher systemic microbial richness and diversity. This surge in systemic microbiota which is sourced from the gut microbiome comes along with a concomitant increase in systemic levels of bacterial biomarkers such as bacterial lipopolysaccharides (LPS) and D-lactate. These products of intestinal bacteria translocation are associated with diminished left ventricular function and an enhancement in inflammation-both of which significantly impact long-term survival and overall outcomes. Leakage of the gut microbiome into the systemic circulation is primarily due to intestinal hyperpermeability caused as a result of diminished LV function and reduced intestinal perfusion post-MI.

Past studies have demonstrated the ability of facultative anaerobic gut bacteria such as E. coli Nissle 1917 (EcN) to colonize and proliferate hypoxic regions of the body such as solid tumors after systemic introduction. Obstruction of the normal cardiac blood flow due to MI creates a similar region of hypoxia and necrosis in the myocardium. Due to the gut microbiome's critical role in the pathophysiology of myocardial infarction, attempts have been made to modulate the gut microbiota to help in the amelioration of MI. Abrogation of the gut microbiome using antibiotics has been tried by multiple groups but has yielded conflicting results. For example, Zhou et al. reported that suppression of gut microbial translocation by removal of gut bacteria using polymyxin B resulted in reduced infarct size, reduced local inflammation and subdued monocyte infiltration. However, Tang et al. reported drastic mortality in mice treated with antibiotics for gut microbiota depletion and emphasized on the role of gut microbiota-derived single chain fatty acids (SCFAs) in conserving host immune response and offering cardioprotection. Lam et al. were the first to use bacteria to target MI where they used commercially available probiotic, Lactobacillus plantarum 299v which is known to reduce serum leptin levels. L. plantarum did afford protection by reducing infarct size, but the results were not significantly different from mice given just antibiotic treatment.

Persistent, long-term inflammation is a major cause for heart failure and progressive ventricular dilatation post-MI. Systemic leakage of bacterial LPS can lead to activation of toll-like receptor 4 which can result in adverse post-infarct maladaptive left ventricular remodeling and impaired cardiac function. Clinical studies have demonstrated the significant influence of LPS on the cardiovascular system through inflammatory and immune pathways.

### Probiotic Bacterial Construct

E. coli is a pioneer of the human intestines—one of the first bacteria to inoculate the human gut—which is ubiquitously present in the guts of over 90% of the population. Facultative anaerobes such as E. coli Nissle 1917 have the ability to survive and prosper in both aerobic as well as anaerobic environments. Numerous studies have demonstrated the ability of intravenously administered EcN to colonize and proliferate in regions of hypoxia. As a result, several attempts have been made to target solid tumors (which have hypoxic and necrotic cores) with EcN. The present invention explored whether this phenomenon of cardiac translocation of EcN could be potentially due to bacteria colonizing in hypoxic regions in the myocardium.

We simulated systemic permeation of bacteria by administering 5×10⁵ cfus of EcN-Lux (a tracer probiotic bacterium producing bioluminescence and GFP) intravenously which resulted in a clear bioluminescent signal from the cardiac region in MI mice, but not shams. This observation was further confirmed by siderophore-based PET imaging and immunohistochemistry resulting in the discovery of this novel phenomenon.

Based on the results, a probiotic bacterial construct was created that reduces bacterial systemic permeation. Gut-derived low-grade endotoxemia has been a major cause of concern in cardiovascular disease resulting in exacerbation of atherosclerosis, thrombosis, and other cardiovascular events. In a clinical study involving 150 subjects found a higher presence of LPS in the coronary thrombi of patients of ST-elevation myocardial infarction (STEMI) versus in the intracoronary blood of stable angina patients. In another clinical study spanning almost 3 years consisting of 912 patients with atrial fibrillation, presence of bacterial lipopolysaccharides (LPS) in the systemic circulation was predictive of major adverse cardiovascular events (MACE) with no association between LPS and classical CV risk factors. The source of this circulating LPS is generally due to increased intestinal permeability which is caused by a variety of lifestyle and disease factors including STEMI. We perceived prevention or amelioration of intestinal hyperpermeability to be an ideal target for our construct.

Faccalibacterium prausnitzii is an important resident of the gut microbiome which is known to produce gut protective metabolites. Microbial anti-inflammatory molecule (MAM) is a small 15 kDa protein which is secreted by Faccalibacterium prausnitzii. F. prausnitzii is a strictly anaerobic bacteria, the intestinal population of which is inversely correlated with gut disorders. MAM has been found to be one of the prominent metabolites behind F. prausnitzii's gut protective actions. MAM exhibits potent anti-inflammatory activity and has been effective in treating colitis as well as restoring intestinal integrity in diabetic mice through the modulation of tight junction transmembrane protein. In the present invention, EcN producing MAM was developed to help reduce post-MI systemic permeation of bacteria by protecting the gut barrier. Using a live biotherapeutic such as EcN is advantageous because it allows for sustained, in situ production of therapeutics in the gut resulting in prolonged action.

### EcN-MAM

As shown in the results below, MI mice given EcN-MAM had significantly less heart to body weight ratio and cardiac fibrosis compared to mice given EcN-Lux, a control bacterium representing natural resident E. coli. EcN-MAM treated mice also showed significantly better echocardiographic parameters indicating enhanced cardiovascular function 28 days post-MI surgery. Following an acute MI event, myocardial cell injury and death results in the onset of an acute pro-inflammatory response which is caused by the combined action of reactive oxygen species production, activation of the complement cascade, and release of damage-associated molecular patterns (DAMPs). The concerted action of neutrophils, cytokines, inflammasomes, toll-like receptor activation, chemokines, MI and Ly-6chigh macrophages, etc. is involved in this phase of post-MI pathophysiology. This pro-inflammatory phase lasts approximately 3 days and is followed by an anti-inflammatory reparative phase. Perturbations in the transition between the two phases often result in a prolonged or excessive pro-inflammatory response resulting in the fibrosis of healthy tissue and poorer overall outcomes. The data evaluating therapeutic efficacy revealed an obvious amelioration in terms of overall outcomes.

MI mice given EcN-MAM showed significantly reduced activation of proinflammatory cells compared to control mice given EcN-Lux or PBS. Without being bound by theory, it was theorized that this could be due to diminished immune activation of mice as a result of reduced systemic permeation of bacteria. Immunoblotting of colon tissue was performed and it was found that EcN-MAM-treated mice had significantly higher levels of intestinal occludin and ZO-1, indicating enhanced gut integrity which has been correlated with significantly lower serum lipopolysaccharide levels. As previously discussed, increased serum LPS level is a common feature in patients of cardiovascular disease. LPS is a potent inducer of toll-like receptor 4 (TLR4)-mediated inflammatory signaling and numerous studies have demonstrated the deleterious effect of TLR4 activation on cardiomyocytes, myocardial injury, and MACE. This metabolic endotoxemia results in chronic systemic inflammation particularly due to the activation of TLR4 and ultimately leads to cardiac fibrosis and heart failure. A significant association between circulating LPS levels observed in the acute phase of an MI event and major adverse cardiovascular events (MACE) has also been noted. Furthermore, a clinical study comprising of 2568 community-dwelling Japanese participants revealed that high serum lipopolysaccharide binding protein (LBP)—an acute phase protein produced by our body in response to LPS—is significantly associated with development of cardiovascular disease.

An ELISA was performed to assess the levels of systemic LBP in MI mice as a surrogate and more reliable marker for systemic LPS. It was observed that EcN-MAM treated mice had significantly lower levels of serum LBP compared to mice given control EcN. Also, depletion of the intestinal microbiome through administration of antibiotic cocktails resulted in a very significant decline in serum LBP levels corroborating the role of gut bacteria as a cause for elevated LBP numbers. An ELISA was also performed for C-reactive protein (CRP) to investigate the state of systemic inflammation in the MI mice. CRP, an acute phase reactant protein and a gold standard of inflammation in the body, has also been found to predict in-hospital outcome and long-term mortality. A clinical study by Mani et al. with 5145 patients revealed significant and independent associations between high sensitivity CRP (hsCRP) and major adverse cardiac event (MACE), cardiovascular death, and all-cause death. Additionally, systemic inflammation has been found to be independently related to 1-year mortality post-MI. It was found that mice treated with EcN-MAM had significantly lower levels of serum CRP compared to control mice and antibiotic cocktail mediated microbial depletion demonstrated a further decrease in serum CRP. Depletion of the gut microbiome has been investigated as a way to mitigate gut bacteria-mediated post-MI systemic inflammation, however, as discussed earlier, this approach has yielded mixed results. Additionally, depletion of the gut microbiome using a cocktail of broad-spectrum antibiotics is not a viable strategy clinically due to risk of antibiotic resistance, superinfections, potential drug-drug interactions and increased side effects.

Oral delivery of protein, peptide, and biological drugs is a significant bottleneck in the field of pharmaceutics and drug delivery. By engineering bacteria to synthesize therapeutic protein-based therapeutics, numerous initial barriers will be circumvented that hamper the oral delivery of molecules belonging to these classes. Achieving therapeutic benefit to distant organs and organ systems using probiotic drug factories present in the gut would be an exciting outcome that would allow the development of numerous therapeutics based on this novel platform. Also, post-infarct pathophysiological changes can be exploited to our benefit by colonizing the gut with recombinant bacteria producing molecules of therapeutic interest.

The present invention takes the novel approach of using a recombinant probiotic platform secreting a defined therapeutic protein, MAM, to target intestinal hyperpermeability and protect against excessive systemic permeation due to systemic bacterial permeation. In one embodiment of the present invention, probiotic E. coli Nissle 1917 expressing F. prausnitzii-derived MAM protein is used to provide sustained, in situ production of gut protective anti-inflammatory proteins.

### Methods

### Engineering MAM-Producing Commensal Bacteria

A bacterial construct was engineered based on E. coli Nissle 1917 (EcN)-expressing F. prausnitzii-derived microbial anti-inflammatory molecule (MAM). Sequence for the protein was obtained from previously published studies. The sequences were codon-optimized for E. coli to allow maximum protein translation. A 6×His tag was added to the protein sequence for easy identification and purification of the protein. Double-stranded gene fragments obtained from Integrated DNA Technologies, Inc. were amplified using polymerase chain reaction (PCR) and incorporated into pBba19A plasmid backbone under the control of J23105 constitutive promoter using standard molecular biology techniques. Bacteria were transformed using heat shock, and plasmids of transformants were extracted. Sanger sequencing was performed on the extracted plasmids to verify their genetic sequence and confirm the incorporation of the gene of interest in the plasmid.

### Bacteria

Recombinant E. coli Nissle 1917 (EcN) producing GFP and luciferase/luciferin was engineered using the pAKgfplux1 plasmid (Addgene plasmid #14083). E. coli Nissle 1917 (EcN) was engineered to produce Fecalibacterium prausnitzii-derived microbial anti-inflammatory protein (MAM). Double-stranded DNA fragment encoding for MAM protein was obtained from Integrated DNA Technologies, Coralville, IA. A 6×His tag was added to the DNA sequence to aid in downstream studies. Fragments were amplified using PCR and ligated into a pBbA19a backbone. Chemically competent EcN were transformed using heat shock and transformed colonies were grown and their sequence was corroborated using Sanger sequencing.

### Establishing the In Vivo Efficacy of Engineered Bacteria

A tracer bacterium, E. coli Nissle 1917 expressing pAKgfplux1 plasmid, was used for the present studies. This plasmid allowed the bacterium to express GFP. Myocardial infarction was induced in C57 mice via left anterior descending artery ligation. Sham surgery was performed on an equal number of mice. Blood was extracted at 24 and 48 h followed by which immunoblotting was performed on serum samples using anti-GFP antibody. Immunoblotting results indicated presence of GFP protein in the serum 24 h and 48 h post-MI.

In another study, eight mice underwent MI surgeries. Four were given EcN expressing MAM plasmid and four were given EcN expressing pAKgfplux1 plasmid. Mice were euthanized 28 days post-surgery and their heart and body weights were measured. Masson's trichome staining, a gold standard stain for cardiac fibrosis, was also performed on heart tissues.

### Myocardial Infarction Procedure

Eight to ten weeks old male C57/BL6 mice were shaved around the thoracic and neck regions and administered sustained-release buprenorphine subcutaneously as per weight (1 mg/kg body weight). The mice were then placed under general anesthesia using isoflurane and put on ventilator. Subsequently, the thorax was opened, pericardial sac cut, and the left anterior descending artery (LADA) was ligated using an 8-0 Prolene suture. The mice were sutured up, ventilator removed, and allowed to recover on a heated pad with oxygen supplementation for half an hour. The sham surgery for this model involved all of the above steps including passing the suture around LADA however the artery itself was not ligated. This creates an excellent control for our model as the sham mice undergo all the surgical procedures as their experimental counterparts except the LADA ligation.

### DNA Extraction and Polymerase Chain Reaction (PCR) Studies

Mouse hearts were extracted aseptically after euthanasia and DNA extraction was carried out using the DNEasy® Blood & Tissue Kit (Qiagen, Germantown, MD) as per the manufacturer's instructions. PCR was performed using DreamTaq® DNA polymerase (ThermoScientific™, Waltham, MA) for a 500 bp fragment of the luxA gene with primers from Integrated DNA Technologies, Coralville, IA. Gel electrophoresis of the PCR product was performed on a 1.5% agarose gel.

For microbiome analysis, each mouse was placed in a sterile cage and their feces were collected. Mice were subsequently euthanized by CO2 inhalation, aseptically dissected, and heart and intestinal tissues were extracted. Heart and intestinal DNA was extracted using Molzym Ultra-Deep Microbiome Prep Kit (Molzym GmbH & Co. KG, Bremen, Germany). Fecal DNA was extracted using QIAamp PowerFecal Pro DNA Kit (Qiagen, Germantown, MD). The extracted DNA was submitted to the Cincinnati Children's Hospital Medical Center DNA Core facility for 16S metagenomics sequencing. Briefly, quality control of DNA samples was performed using 515F-806R primers. The V4 hypervariable region of the 16S rRNA gene was amplified and sequenced as per Illumina MiSeq's 250PE run protocol. The generated data was analyzed using standard QIIME2 protocols (Bolyen et al., 2019).

### EcN Gut Colonization Model

Mouse drinking water was supplemented with 0.25 mg/mL ampicillin. Mice were given 108 colony forming units (cfu) of log-phase E. coli Nissle 1917. Bacteria were given once every week.

### Histopathology

After euthanasia via carbon dioxide inhalation, mouse hearts were isolated, weighed, and fixed in neutral buffered formalin (NBF) and embedded in paraffin. 6-micron sections of the tissue were cut by Cincinnati Children's Hospital Medical Center Department of Pathology Research Core (Cincinnati, OH, USA). The sections were stained for Masson's trichome to assess post-MI fibrosis. Immunohistochemistry was performed using anti-GFP antibody (Abcam).

### Immunoblotting to Confirm Bacterial Synthesis of MAM Protein

Overnight culture of EcN-MAM was inoculated in 100 mL of terrific broth (1:100 dilution) and incubated at 37 C at 200 rpm in a shaker-incubator for 3 hours. Subsequently, the culture was centrifuged at 3000 g for 15 min at 4 C, followed by careful removal of the supernatant. To allow for efficient protein extraction, the bacterial pellet was frozen in dry ice for 1 h and then allowed to thaw on ice. Bacterial protein was extracted using Bacterial Protein Extraction Reagent (B-PER™) supplemented with DNase I, lysozyme, and Halt™ protease inhibitor cocktail as per the manufacturer's instructions. The extracted protein was mixed with Tricine Sample Buffer (BioRad, Hercules, CA) as per manufacturer's instructions and denatured at 95 C for 10 min.

The protein samples were loaded into a 16.5% tris-tricine gel and SDS-PAGE was performed in a tris/tricine/SDS buffer system (Biorad, Hercules, CA). The gel was transferred onto a 0.2 μm nitrocellulose membrane and blocked using 5% bovine serum albumin (BSA) in tris-buffered saline (TBS). The membrane was then incubated with HRP conjugated 6×His-tag monoclonal antibody (Proteintech, Rosemont, IL) at 1:10,000 dilution in TBS-Tween. Membrane was washed three times with TBS-Tween followed by addition of Supersignal™ West Pico PLUS Chemiluminescent Substrate (Thermo Scientific, Waltham, MA). The blot was detected using the C-DiGit Blot Scanner (LI-COR Biosciences, Lincoln, NE).

### Immunoblotting for Intestinal Tight Junction Proteins

About 5 mm of tissue from the large intestine prior to cecum was dissected and flash frozen. Total protein was extracted by homogenization in RIPA buffer supplemented with Halt™ protease inhibitor cocktail. Protein levels were normalized, and samples were loaded into a 12% tris-glycine gel and SDS-PAGE was performed in a tris/glycine/SDS buffer system (Biorad, Hercules, CA). The gel was transferred to a 0.45 μm PVDF membrane, blocked in 5% milk-TBS-Tween and incubated with primary antibodies (Proteintech, Rosemont, IL) at manufacturer recommended dilutions for 2 hours. Membrane was washed three times with TBS-Tween, followed by incubation with relevant HRP-conjugated secondary antibody (Proteintech). After three subsequent washes with TBS-Tween, the membrane was developed using Supersignal™ West Pico PLUS Chemiluminescent Substrate (Thermo Scientific, Waltham, MA). The blot was detected using the C-DiGit Blot Scanner (LI-COR Biosciences, Lincoln, NE).

### Echocardiography Studies

Cardiac function was assessed via echocardiography using a Vevo 2100 imaging system (VisualSonics, Toronto, Canada). Mice were anesthetized using isoflurane inhalation, chest hair were removed using a depilatory cream, and each mouse was subsequently placed on a heated platform maintained at 38° C. with paws in contact with the electrocardiography leads. Mouse core body temperature was monitored using a rectal probe and maintained at 36-37° C. using a heat lamp and the heated platform. M-mode echocardiography was performed at the parasternal long axis and images were analyzed for various parameters of cardiac function such as cardiac output, fractional shortening, ejection fraction, and LV mass. Mice were imaged pre- and post-surgery followed by weekly electrocardiography imaging. Post euthanasia, mouse tibial lengths and heart weights were determined for normalization and assessment of fibrosis and hypertrophy.

Analysis of Inflammation-Associated Genes Using Nanostring nCounter

NanoString nCounter (NanoString Technologies) was used to assess the impact of our anti-inflammatory therapy on the MI heart. MI mice were treated with either EcN-pAKgfplux1 or EcN-MAM. Two days post-surgery, mice were euthanized, hearts were isolated and promptly transferred to RNAlater Tissue Storage Reagent (ThermoFisher, Waltham, MA) and flash frozen in liquid nitrogen. The samples were submitted to Canopy Biosciences (St. Louis, MO) for further processing. Tissue including and surrounding the infarct region of the heart (Apex) was cut and RNA was extracted using Takara Nucleospin RNA kit (Takara Bio, San Jose, CA) as per manufacturer's protocol. RNA quantification was done using a NanoDrop 2000 spectrophotometer while quality was assessed using Agilent 2100 and 100 ng of RNA was used as input to the NanoString hybridization. RNA was analyzed using the nCounter Mouse Inflammation Panel (NanoString) which comprised of 254 genes associated with inflammation. Hybridization assay was performed on the nCounter MAX system and the generated raw data was analyzed by Canopy Biosciences using Rosalind (www.rosalind.bio, San Diego, CA).

### Flow Cytometry

Perfused and harvested hearts were minced into 1 mm pieces using a razor blade. The minced hearts were washed three times with phosphate-buffered saline (PBS), as previously described (ref), to remove intravascular cells. The hearts were then digested for 45 minutes at 37° C. under agitation, in Hank's Balanced Salt Solution (HBSS) solution containing 1 mg/ml Collagenase IV (Gibco) and 10 ug/ml DNase I (Sigma-Aldrich). Cell suspensions were filtered through a 70 μm strainer, and debris were removed using Debris removal solution (Miltenyi), following the supplier's protocol. For immunophenotyping by flow cytometry, two million cells per sample were used. The single cell suspensions were first labeled for 30 minutes at 4° C. with LIVE/DEAD Fixable Blue (Invitrogen), to separate live cells from dead cells during analysis. After two washes, the samples were incubated for 30 minutes at 4° C. with the following antibodies: anti-CD45-BUV496 (BD Bioscience, 30-F11), Anti-CD3-APC (Biolegend, 17A2), anti-CD11b-PacificBlue (Biolegend, M1/70), anti-CD8-PE (Biolegend, 53-6.7), anti-CD4-BV785 (Biolegend, RM4-5), anti-CD19-BV785 (Biolegend, 6D5), anti-Ly6G-PE (Biolegend, 1A8), anti-NK1.1-PeCy7 (Biolegend, PK136), anti-MHCII-FITC (Biolegend, M5/114.15.2), anti-CD16/32-PerCP-Cy5.5 (BD Bioscience, 2.4G2), anti-SiglecF-PE-Efluor610 (eBioscience, 1RNM44N), anti-Ly6C-AlexaFluor700 9Biolegend, HK1.4), and anti-CD86-APC-Cy7 (Biolegend, GL-1). Following two washes with PBS, the samples were fixed and permeabilized using the Foxp3 staining kit (eBioscience), according to the manufacturer's protocol. After permeabilization, the samples were incubated with anti-CD68-BV421 (BD Bioscience, FA/11) for 30 minutes at 4° C. Samples were acquired on an Aurora full spectrum cytometer (Cytek) in the Cincinnati Children's Hospital medical center Flow cytometry core. Unstained and single-stained samples were acquired and used to calculate the unmixing parameter using Spectroflow (Cytek). Unmixed samples files were exported as FCS files and analyzed using FlowJo software (BD Bioscience).

### ELISA

ELISA for serum lipopolysaccharide binding protein was performed with mouse serum using Mouse LBP ELISA Kit PicoKine™ (BosterBio, Pleasanton, CA) as per manufacturer's instructions. Serum was obtained by allowing mouse blood to clot at room temperature for 30 min followed by refrigerated centrifugation at 2000×g for 15 min.

ELISA for CRP was performed using Mouse C-Reactive Protein/CRP ELISA Kit from ProteinTech, Rosemont, IL as per manufacturer's instructions using mouse serum.

### EXAMPLES

### Example 1

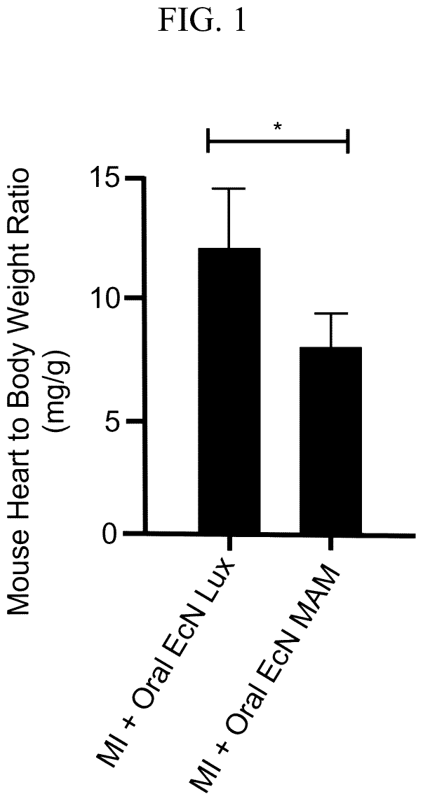

Bacteria containing the gene responsible for the production of MAM were successfully engineered. Sanger sequencing of transformed colonies confirmed incorporation of MAM genes into the pBba19a plasmid. The heart weight to body weight ratio, which is a well-established parameter of cardiac hypertrophy and fibrosis, was determined for each mouse. Results revealed that mice given EcN pAKgfplux1 had a significantly higher heart to body weight ratio compared to mice given EcN MAM indicating higher fibrosis and hypertrophy (see FIG. 1).

### Example 2

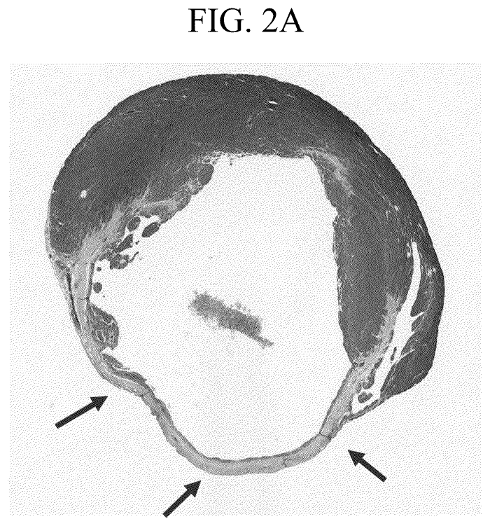

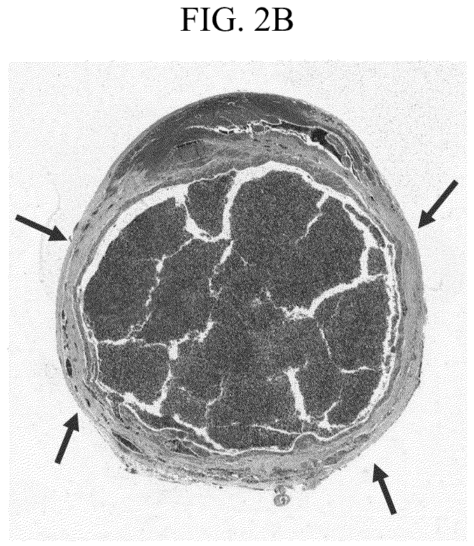

Masson's trichrome staining is commonly used to assess the extent of fibrotic tissues in the heart. Masson's trichrome staining was performed 28 days post MI surgery to assess and compare cardiac hypertrophy in treated vs. untreated controls. Staining images indicated that mice which were given EcN MAM had significantly lower fibrosis compared to mice given control EcN (see FIGS. 2A and 2B). These results indicate that MAM is effective in ameliorating myocardial infarction-associated fibrosis and cardiac hypertrophy.

### Example 3

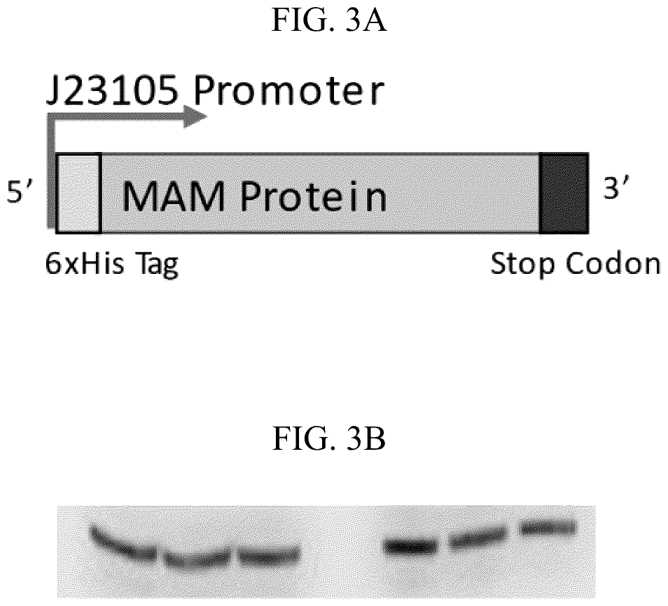

EcN was engineered to produce F. prausnitzii-derived MAM protein. A constructed pBbA19a plasmid containing 6×His tagged MAM protein was transformed into EcN using heat shock and transformants were obtained using ampicillin selection (FIG. 3A). Sanger sequencing of transformed colonies confirmed successful incorporation of pBbA19a-MAM plasmid into EcN. Immunoblotting of EcN-MAM was performed to validate the synthesis of MAM protein by the bacteria. SDS-PAGE of bacterial protein followed by immunoblotting using anti-6×His Tag antibody demonstrated presence of MAM in the bacterial protein extract (FIG. 3B).

### Example 4

Previous studies have reported MAM to have potent anti-inflammatory action. It has also shown to restore gut integrity after a pathological insult. Mice were given either EcN-MAM or EcN-Lux and MI surgeries were performed. Mice were euthanized 28 days post-surgery and heart weight to body weight ratio (HWBWR) was determined. Furthermore, Masson's trichrome staining—the gold standard for assessing cardiac fibrosis—was performed on the heart tissues. It was found that mice given EcN-MAM had a significantly lesser HWBWR compared to mice given EcN-Lux indicating lower fibrosis and hypertrophy and overall better cardiac healing. Masson's trichrome staining of the infarct heart showed significantly lower presence of fibrotic tissue in mice given EcN-MAM vs. those given EcN-Lux. Quantitation of fibrotic staining showed significantly lower fibrosis in EcN-MAM treated mice. These results indicate that treatment with EcN-MAM reduces post-MI cardiac fibrosis and hypertrophy.

### Example 5

A myocardial infarction event results in left ventricular remodeling (LV remodeling) which is denoted by functional and geometric transformations in the left ventricle resulting in the dilatation of the tissue in the infarcted region and hypertrophy in the unaffected myocardial segments. LV remodeling results in diminished LV ejection fraction and is associated with inferior clinical outcomes (Ong et al., 2018). Echocardiography was performed to assess various parameters of cardiac function in mice treated with EcN MAM. It was found that mice treated with EcN MAM had significantly better ejection fraction, fractional shortening, LV volume; d, and LV volume; s values compared to untreated, and EcN Lux treated mice.

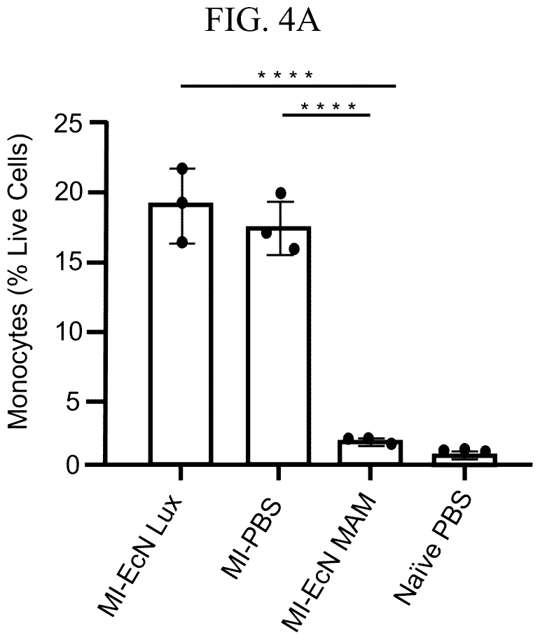

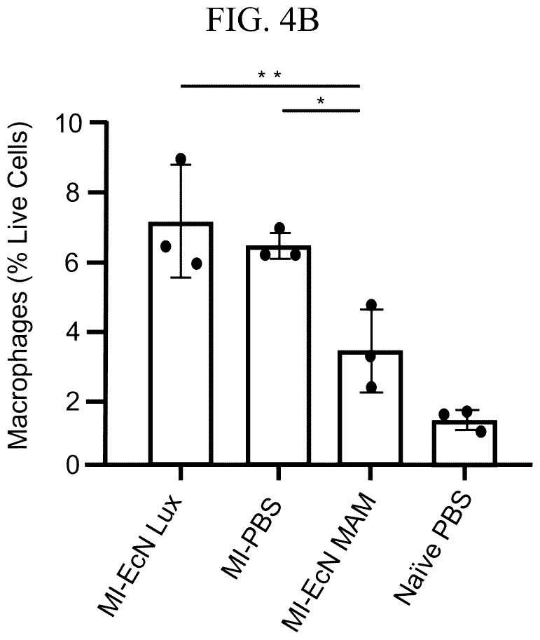

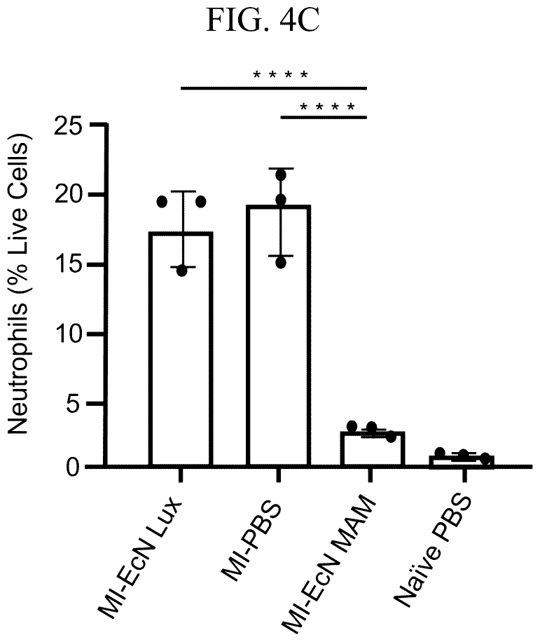

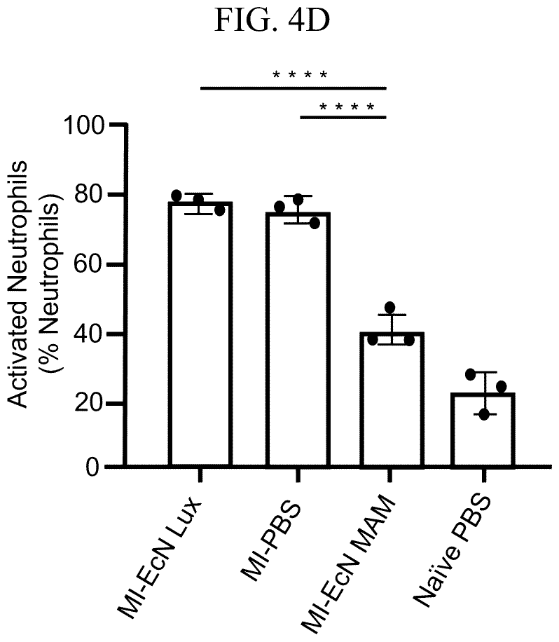

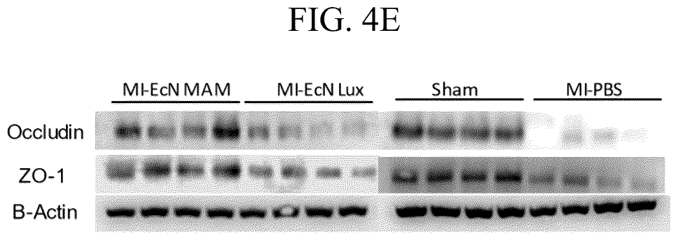

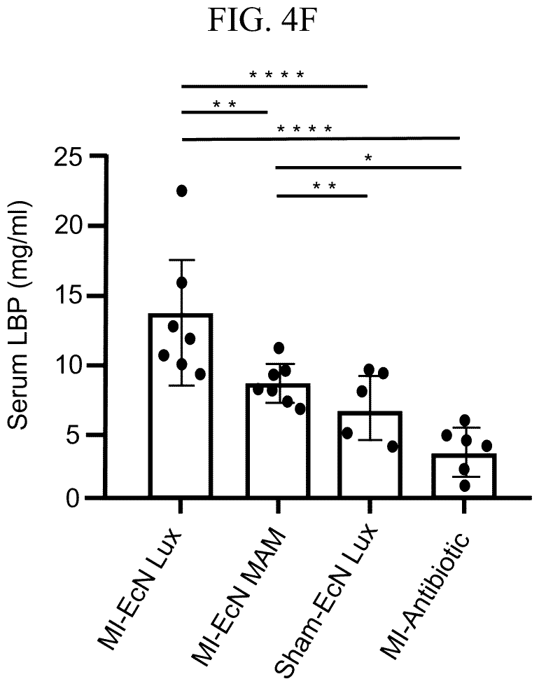

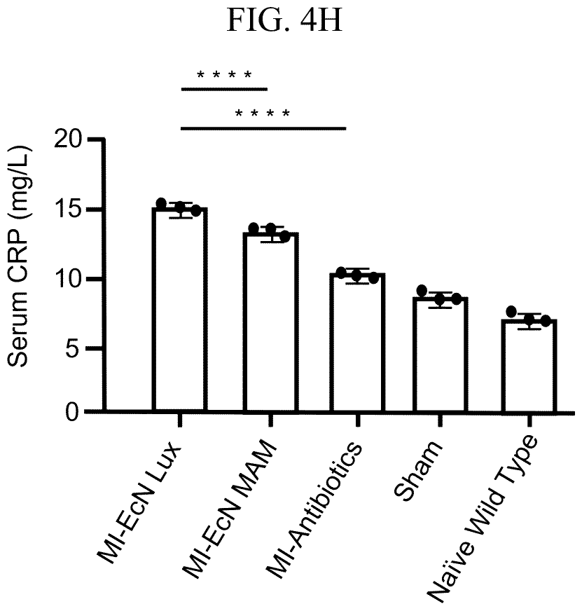

Additionally, flow cytometry was performed to evaluate immune changes in the hearts of EcN MAM-treated mice at 48 h post MI. As indicated by the UMAP images, there was a clear imbalance in the proportion of immune cells compared to the naïve control with expected infiltration of monocytes and neutrophils in the MI groups. Further analysis of immune cell population indicates that EcN MAM-treated group had significantly less numbers of monocytes, macrophages, neutrophils, and activated neutrophils when represented in percent of total live cells (FIGS. 4A-4D). This indicates a significantly lower proinflammatory response in EcN-MAM treated mice as a result of lower systemic bacterial permeation and subsequent response by the immune system.

### Example 6

The Fecalibacterium prausnitzii-derived MAM protein (SEQ. 1) has been known to protect and restore the gut barrier. As previously discussed, gut barrier integrity is significantly affected by an MI event resulting in intestinal hyperpermeability. Immunoblotting was performed against intestinal tight junction proteins-occludin and zona occludens-1 (ZO-1) on protein extracted from the large intestinal tissue dissected 48 h after surgery. It was found that mice dosed with EcN-MAM had higher gut barrier integrity as revealed by higher occludin and ZO-1 staining compared to mice treated with EcN Lux and untreated mice (FIG. 4E).

As demonstrated by previous studies, intestinal hyperpermeability results in the leakage of live bacteria, bacterial cellular components, and by-products into the systemic circulation. This introduction of bacteria in blood results in endotoxemia and inflammatory response. Lipopolysaccharide binding protein (LBP) is produced in response to LPS and plays a crucial role in the inflammatory response by the innate immune system. Serum levels of LBP in the systemic circulation of mice were evaluated 48 h post-surgery. It was observed that mice treated with EcN MAM had significantly low levels of serum LBP compared to those treated with EcN Lux. Increased presence of serum LBP indicates a higher innate immune system-mediated inflammatory response (FIG. 4F).

### Example 7

C-reactive protein (CRP) is a well-established marker of systemic inflammation. Murine CRP, although not as sensitive as its human counterpart, provides a good indication of the post-MI inflammatory response. Our results show that EcN-MAM treated mice had significantly lower CRP-levels compared to EcN-Lux mice. It was also found that treatment with a cocktail of broad-spectrum antibiotics resulted in significantly lower CRP which further corroborates the gut bacteria-mediated inflammation hypothesis (FIG. 4G).

The embodiments of the present invention recited herein are intended to be merely exemplary and those skilled in the art will be able to make numerous variations and modifications to it without departing from the spirit of the present invention. Notwithstanding the above, certain variations and modifications, while producing less than optimal results, may still produce satisfactory results. All such variations and modifications are intended to be within the scope of the present invention as defined by the claims appended hereto.

### Sequences

Sequences for the MAM protein (SEQ. 1) and EcN-MAM plasmid (SEQ. 2) are disclosed.

SEQ. 1:
ATGATGATGCCTGCAAACTACTCTGTTATCGCAGAGAACGAAATGACCTA
CGTCAACGGTGGCGCTAACTTCATCGACGCTATCGGCGCTGTTACCGCTC
CTATCTGGACTCTGGACAACGTTAAGACCTTCAACACCAACATCGTGACT
CTGGTTGGCAACACCTTCCTGCAGTCCACCATTAACCGCACCATCGGTGT
CCTGTTCAGCGGCAACACCACCTGGAAGGAAGTCGGCAACATCGGCAAGA
ACCTGTTCGGCACCAATGTTAAGGGCAACCCGATCGAGAAGAACAACTTT
GGTGACTATGCTATGAACGCTCTGGGCATTGCTGCTGCTGTCTACAACCT
GGGCGTGGCTCCCACCAAGAACACCGTCAAGGAGACTGAGGTTAAGTTCA
CTGTCTAA
SEQ. 2:
TTACCAATGCTTAATCAGTGAGGCACCTATCTCAGCGATCTGTCTATTTC
GTTCATCCATAGTTGCCTGACTCCCCGTCGTGTAGATAACTACGATACGG
GAGGGCTTACCATCTGGCCCCAGTGCTGCAATGATACCGCGAGACCCACG
CTCACCGGCTCCAGATTTATCAGCAATAAACCAGCCAGCCGGAAGGGCCG
AGCGCAGAAGTGGTCCTGCAACTTTATCCGCCTCCATCCAGTCTATTAAT
TGTTGCCGGGAAGCTAGAGTAAGTAGTTCGCCAGTTAATAGTTTGCGCAA
CGTTGTTGCCATTGCTACAGGCATCGTGGTGTCACGCTCGTCGTTTGGTA
TGGCTTCATTCAGCTCCGGTTCCCAACGATCAAGGCGAGTTACATGATCC
CCCATGTTGTGCAAAAAAGCGGTTAGCTCCTTCGGTCCTCCGATCGTTGT
CAGAAGTAAGTTGGCCGCAGTGTTATCACTCATGGTTATGGCAGCACTGC
ATAATTCTCTTACTGTCATGCCATCCGTAAGATGCTTTTCTGTGACTGGT
GAGTACTCAACCAAGTCATTCTGAGAATAGTGTATGCGGCGACCGAGTTG
CTCTTGCCCGGCGTCAATACGGGATAATACCGCGCCACATAGCAGAACTT
TAAAAGTGCTCATCATTGGAAAACGTTCTTCGGGGCGAAAACTCTCAAGG
ATCTTACCGCTGTTGAGATCCAGTTCGATGTAACCCACTCGTGCACCCAA
CTGATCTTCAGCATCTTTTACTTTCACCAGCGTTTCTGGGTGAGCAAAAA
CAGGAAGGCAAAATGCCGCAAAAAAGGGAATAAGGGCGACACGGAAATGT
TGAATACTCATACTCTTCCTTTTTCAATATTATTGAAGCATTTATCAGGG
TTATTGTCTCATGAGCGGATACATATTTGAATGTATTTAGAAAAATAAAC
AAATAGGGGTTCCGCGCACATTTCCCCGAAAAGTGCCACCTGACGTCTTT
ACGGCTAGCTCAGTCCTAGGTACTATGCTAGCAGGTTTAATCGAATTCAA
AAGATCTTTTAAGAAGGAGATATACATATGTGACTCGAGATGAAAATCAT
GCACCATCACCACCACCACATGATGATGCCTGCAAACTACTCTGTTATCG
CAGAGAACGAAATGACCTACGTCAACGGTGGCGCTAACTTCATCGACGCT
ATCGGCGCTGTTACCGCTCCTATCTGGACTCTGGACAACGTTAAGACCTT
CAACACCAACATCGTGACTCTGGTTGGCAACACCTTCCTGCAGTCCACCA
TTAACCGCACCATCGGTGTCCTGTTCAGCGGCAACACCACCTGGAAGGAA
GTCGGCAACATCGGCAAGAACCTGTTCGGCACCAATGTTAAGGGCAACCC
GATCGAGAAGAACAACTTTGGTGACTATGCTATGAACGCTCTGGGCATTG
CTGCTGCTGTCTACAACCTGGGCGTGGCTCCCACCAAGAACACCGTCAAG
GAGACTGAGGTTAAGTTCACTGTCTAATGAAAGCTTGGATCCAAACTCGA
GTAAGGATCTCCAGGCATCAAATAAAACGAAAGGCTCAGTCGAAAGACTG
GGCCTTTCGTTTTATCTGTTGTTTGTCGGTGAACGCTCTCTACTAGAGTC
ACACTGGCTCACCTTCGGGTGGGCCTTTCTGCGTTTATACCTAGGGATAT
ATTCCGCTTCCTCGCTCACTGACTCGCTACGCTCGGTCGTTCGACTGCGG
CGAGCGGAAATGGCTTACGAACGGGGCGGAGATTTCCTGGAAGATGCCAG
GAAGATACTTAACAGGGAAGTGAGAGGGCCGCGGCAAAGCCGTTTTTCCA
TAGGCTCCGCCCCCCTGACAAGCATCACGAAATCTGACGCTCAAATCAGT
GGTGGCGAAACCCGACAGGACTATAAAGATACCAGGCGTTTCCCCCTGGC
GGCTCCCTCGTGCGCTCTCCTGTTCCTGCCTTTCGGTTTACCGGTGTCAT
TCCGCTGTTATGGCCGCGTTTGTCTCATTCCACGCCTGACACTCAGTTCC
GGGTAGGCAGTTCGCTCCAAGCTGGACTGTATGCACGAACCCCCCGTTCA
GTCCGACCGCTGCGCCTTATCCGGTAACTATCGTCTTGAGTCCAACCCGG
AAAGACATGCAAAAGCACCACTGGCAGCAGCCACTGGTAATTGATTTAGA
GGAGTTAGTCTTGAAGTCATGCGCCGGTTAAGGCTAAACTGAAAGGACAA
GTTTTGGTGACTGCGCTCCTCCAAGCCAGTTACCTCGGTTCAAAGAGTTG
GTAGCTCAGAGAACCTTCGAAAAACCGCCCTGCAAGGCGGTTTTTTCGTT
TTCAGAGCAAGAGATTACGCGCAGACCAAAACGATCTCAAGAAGATCATC
TTATTAATCAGATAAAATATTTCTAGATTTCAGTGCAATTTATCTCTTCA
AATGTAGCACCTGAAGTCAGCCCCATACGATATAAGTTGTTACTAGTGCT
TGGATTCTCACCAATAAAAAACGCCCGGCGGCAACCGAGCGTTCTGAACA
AATCCAGATGGAGTTCTGAGGTCATTACTGGATCTATCAACAGGAGTCCA
AGCGAGCTCGTAAACTTGGTCTGACAG

What is claimed is:

1. A genetically modified E. coli bacterium wherein the bacterium expresses a microbial anti-inflammatory molecule (MAM) protein.

2. The genetically modified E. coli bacterium of claim 1, wherein the bacterium is a probiotic.

3. The genetically E. coli probiotic bacterium of claim 1, wherein the bacterium that is modified is E. coli Nissle 1917.

4. The genetically modified E. coli bacterium of claim 1, wherein the MAM protein has a sequence of [SEQ ID NO: 1].

5. The genetically modified E. coli bacterium of claim 1, wherein the bacterium expresses a plasmid having a sequence of [SEQ ID NO: 2].

6. A pharmaceutical composition comprising the genetically modified E. coli bacterium of claim 1 and a pharmaceutically acceptable excipient.

7. A pharmaceutical composition comprising the genetically modified E. coli bacterium of claim 3 and a pharmaceutically acceptable excipient.

8. A pharmaceutical composition comprising the genetically modified E. coli bacterium of claim 4 and a pharmaceutically acceptable excipient.

9. A pharmaceutical composition comprising the genetically modified E. coli bacterium of claim 5 and a pharmaceutically acceptable excipient.

10. A plasmid comprising the sequence of [SEQ ID NO: 2].

11. A method of reducing cardiac inflammation in a subject who has experienced a myocardial infarction comprising administering a pharmaceutically effective amount of a genetically modified E. coli bacterium to the subject, wherein the bacterium expresses a microbial anti-inflammatory molecule (MAM) protein.

12. The method of claim 10 wherein the bacterium is a probiotic.

13. The method of claim 10 wherein the bacterium that is modified is E. coli Nissle 1917.

14. The method of claim 10 wherein the MAM protein has a sequence of [SEQ ID NO: 1].

15. The method of claim 10 wherein the bacterium expresses a plasmid having a sequence of [SEQ ID NO: 2].

16. The method of claim 10 wherein a pharmaceutical composition comprising the genetically modified E. coli bacterium and a pharmaceutically acceptable excipient is administered to the patient.

17. The method of claim 10 wherein the genetically modified E. coli bacterium is administered orally.


SEQUENCE LISTING (2 sequences)

SEQ ID NO: 1 - DNA, 408 bp, Faecalibacterium prausnitzii
  source 1..408: mol_type = other DNA
atgatgatgcctgcaaactactctgttatcgcagagaacgaaatgacctacgtcaacggtggcgctaacttcatcgacgctatcggcgctgttaccgctcctatctggactctggacaacgttaagaccttcaacaccaacatcgtgactctggttggcaacaccttcctgcagtccaccattaaccgcaccatcggtgtcctgttcagcggcaacaccacctggaaggaagtcggcaacatcggcaagaacctgttcggcaccaatgttaagggcaacccgatcgagaagaacaactttggtgactatgctatgaacgctctgggcattgctgctgctgtctacaacctgggcgtggctcccaccaagaacaccgtcaaggagactgaggttaagttcactgtctaa

SEQ ID NO: 2 - DNA, 2627 bp, synthetic construct
  source 1..2627: mol_type = other DNA
ttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctttacggctagctcagtcctaggtactatgctagcaggtttaatcgaattcaaaagatcttttaagaaggagatatacatatgtgactcgagatgaaaatcatgcaccatcaccaccaccacatgatgatgcctgcaaactactctgttatcgcagagaacgaaatgacctacgtcaacggtggcgctaacttcatcgacgctatcggcgctgttaccgctcctatctggactctggacaacgttaagaccttcaacaccaacatcgtgactctggttggcaacaccttcctgcagtccaccattaaccgcaccatcggtgtcctgttcagcggcaacaccacctggaaggaagtcggcaacatcggcaagaacctgttcggcaccaatgttaagggcaacccgatcgagaagaacaactttggtgactatgctatgaacgctctgggcattgctgctgctgtctacaacctgggcgtggctcccaccaagaacaccgtcaaggagactgaggttaagttcactgtctaatgaaagcttggatccaaactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttatacctagggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgttactagtgcttggattctcaccaataaaaaacgcccggcggcaaccgagcgttctgaacaaatccagatggagttctgaggtcattactggatctatcaacaggagtccaagcgagctcgtaaacttggtctgacag